(19) United States
(12) Patent Application Publication
(10) Pub. No.: US 2012/0203322 A1
(43) Pub. Date: Aug. 9, 2012

(54) QUICK RELEASE MECHANISM FOR MEDICAL DEVICE DEPLOYMENT

(76) Inventor: Robert M. Eells, Bloomington, IN (US)

(21) Appl. No.: 13/022,055

(22) Filed: Feb. 7, 2011

(51) Int. Cl. A61F 2/84

(52) U.S. Cl. 623/1.11

(57) ABSTRACT

A medical device assembly having a quick release mechanism reversibly engaged with a medical device and a method used by an operator to deploy said medical device at a targeted site in a body vessel is provided. The quick release mechanism generally comprises an inner core wire and an outer coil having a proximal section and distal section. The inner core wire and the outer coil are coupled together proximate to their distal end with the outer coil having an enlarged overall thickness in its distal section. The medical device has an opening sized to receive and to detachably engage the distal section of the outer coil. The quick release mechanism having an engaged position in which the outer coil is securely holds the medical device and a detached position in which the outer coil and medical device are substantially unengaged. The operator causes the quick release mechanism to move from the engaged position to the detached position by moving the inner core wire and outer coil in opposite directions.

### FIELD

This disclosure relates generally to a mechanism for the deployment of a medical device within a patient. More specifically, this disclosure relates to a delivery assembly for a medical device and a method of mechanically releasing or detaching said device at a targeted vascular site.

### BACKGROUND

A standard procedure used in the treatment of endovascular diseases is the placement of medical devices, such as embolic coils, stents, and dilation balloons, among others, at a desired or targeted site within a patient. The delivery of such a medical device have typically been accomplished by a variety of means, including the use of a catheter in which a pusher forces the device through the catheter to be deployed at the targeted site. These medical devices usually have a contracted shape that allows them to pass through the lumen of the catheter and an expanded shape that occurs after being deployed to the targeted site, such as an aneurysm.

One example, of such a medical device is an embolic or occlusive device that is placed within the vasculature of the human body, to filter the flow of blood through a vessel in the vasculature or to block the flow of blood within a defect in the vessel, such as an aneurysm. One widely accepted occlusive device is a helical wire coil whose coil windings are sized to engage the wall of the vessel. In this case, a catheter is first placed at or near the targeted site within the vessel. This catheter may be guided to the targeted site through the use of guide wires or the like. Once the distal end of the catheter has reached the site, one or more helical wire coils are placed into the proximal end of the catheter and advanced through the catheter using the pusher. Once the coil reaches the distal end of the catheter, the pusher discharges it from the catheter.

Despite the technological advancement in the field of delivering such occlusive devices to a target site, problems still exist with many of the current means of deployment. These problems include the ease of positioning and repositioning the helical wire coil before detachment from the catheter, the accuracy in maneuvering the coil into position at the target site, and the duration of time necessary to deploy the coil, to name a few. The inaccurate placement of the coil can be problematic because once the coil has left the catheter, it is difficult to reposition or retrieve the coil. In addition, the use of a pusher to force the coil out of the catheter can result in localized damage to the vasculature, such as thermal damage to the tissue surrounding the distal end of the catheter.

Accordingly, there exists a desire to provide improvements in the mechanism used to detachably deploy a medical device at a targeted location in the vasculature of a patient. More particularly, there exists a desire for the continued development of a coupling mechanism that securely holds the medical device, thereby, allowing it to be effectively maneuvered throughout the deployment process, while also allowing said medical device to be easily and reliably detached once it is properly located at the target site. A mechanism that is adaptable for use with a wide variety of medical devices would be advantageous.

### SUMMARY

In satisfying the above need, as well as overcoming the enumerated drawbacks and other limitations of the related art, the present disclosure generally provides a medical device assembly having a quick release mechanism for use by an operator in deploying a medical device at a targeted site in a body vessel. The medical device assembly comprising a quick release mechanism and a medical device reversibly attached thereto.

According to one aspect of the present disclosure, the quick release mechanism comprises a pusher element having a distal end, the pusher element including an inner core wire and an outer coil. The inner core wire and outer coil are coupled together at the distal end of the pusher element. The outer coil, which is also defined by a proximal section and a distal section, has an enlarged overall thickness in the distal section.

According to another aspect of the present disclosure, the medical device has a proximal part and a distal part with the proximal part including an opening that is sized to receive and to detachably engage the distal section of the outer coil. Movement of the outer coil opposite to the movement of the inner core wire reduces the thickness of the outer coil in the distal section, thereby, allowing the medical device to detach from the wire guide for deployment in the body vessel.

According to yet another aspect of the present disclosure, a method is provided for use by an operator in deploying a medical device at a targeted site in a body vessel. The method generally comprises the steps of introducing a catheter having a distal end and a proximal end into a body vessel. The distal end of the catheter being positioned proximate to a targeted site within the vessel. Then a medical device assembly is placed into the proximal end of the catheter. The medical device assembly comprises a medical device and a quick release mechanism as described herein having an engaged position in which the medical device and quick release mechanism are engaged and a detached position in which the medical device and quick release mechanism are not engaged. The medical device assembly is then moved through the catheter to the targeted site in the vessel while the quick release mechanism is in the engaged position. The operator may cause the quick release mechanism to move from the engaged position to the detached position, thereby, deploying the medical device at the targeted site in the vessel. Optionally, the operator may further position the medical device proximate to the targeted site after the device exits the catheter prior to causing the quick release mechanism to transition to the detached state.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

The drawings described herein are for illustration purposes only and are not intended to limit the scope of the present disclosure in any way.

FIG. 1A is a perspective view of a quick release mechanism in its engaged position prepared according to the teachings of the present disclosure;

FIG. 1B is a perspective view of the quick release mechanism of FIG. 1A shown in its detached position according to another aspect the present disclosure;

FIG. 2A is another perspective view of a quick release mechanism in its engaged position prepared according to the teachings of the present disclosure;

FIG. 2B is a perspective view of the quick release mechanism of FIG. 2A shown in its detached position according to another aspect the present disclosure;

FIG. 3A is a perspective view of a quick release mechanism prepared according to the teachings of the present disclosure highlighting engagement with a medical device;

FIG. 3B is a perspective view of the quick release mechanism of FIG. 3A according to another aspect the present disclosure;

FIG. 4A is another perspective view of a quick release mechanism prepared according to the teachings of the present disclosure highlighting engagement with a medical device;

FIG. 4B is a perspective view of the quick release mechanism of FIG. 4A according to another aspect the present disclosure; and

FIG. 5 is a schematic representation of a method of deploying a medical device into a vasculature of a patient using a quick release mechanism made according to the teachings of the present disclosure.

### DETAILED DESCRIPTION

The following description is merely exemplary in nature and is in no way intended to limit the present disclosure or its application or uses. It should be understood that throughout the description and drawings, corresponding reference numerals indicate like or corresponding parts and features.

The present disclosure generally provides a quick release mechanism for the deployment of a medical device at a targeted site in the vasculature of a patient, as well as a method of using said mechanism. The quick release mechanism basically comprises a pusher element having a wire coiled around the distal end of an inner core wire. The coiled wire has a proximal and distal section and is capable of interacting with a medical device to either hold or release said device in an engaged or detached position, respectively. The quick release mechanism has an engaged position in which the distal section of the coiled wire makes contact with the inner wall of an opening in the medical device to securely hold such device during deployment. The medical device may be attached proximate to the distal end of the coiled wire during the manufacturing process or by the attending operator prior to performing the deployment procedure by placing the distal section of the coiled wire into the opening in the medical device such that the medical device is releasably held by quick release mechanism. In use, the coiled wire, with the medical device attached, is advanced by the pusher element through a catheter to a target vascular site in a patient. Upon exiting the distal end of the catheter, the quick release mechanism is made to undergo a transition to a detached position, in which the coiled wire no longer engages the medical device, thereby, causing the medical device to be released or deployed. The pusher element can then be withdrawn, leaving the medical device in the desired position. Thus, the quick release mechanism of the present disclosure does not automatically release the medical device when it is extruded from the catheter, but rather, requires operator action to switch the mechanism from the coupled or engaged position to its detached or unengaged position.

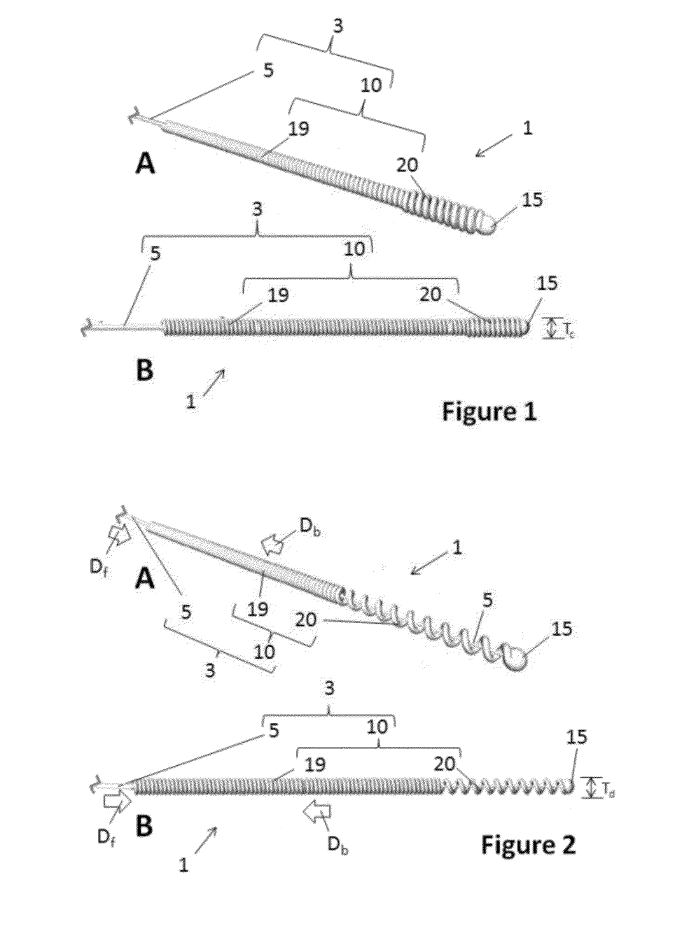

Referring to FIGS. 1A and 1B, the quick release mechanism 1 comprises a pusher element 3 having an inner core wire 5 defined by a proximal and distal portion and a wire 10 coiled around the distal portion of the core wire 5. The inner core wire 5 and the coiled wire 10 are coupled at the distal end 15 of the pusher element 3. The coiled wire 10 also is defined by a proximal 19 and a distal 20 section. The coiled wire 10 proximate to the distal section 20 has a larger diameter than the wire coiled proximate to the proximal section 19. Thus in a relaxed state or engaged position, the overall thickness (T_{c}) of the coiled wire is greatest in its distal section 20.

Referring now to FIGS. 2A & 2B, the quick release mechanism 1 can be switched to a detached position by the inner core wire 5 being moved in the forward or distal direction (D_{f}) relative to the vasculature in the patient, while the coiled wire 10 is pulled backward or moved in the proximal direction (D_{b}). This combined movement causes the wire coiled at the distal section 20 to become extended or stretched. Upon such extension, the overall thickness (T_{d}) of the coiled wire 10 is reduced at the distal section 20. Preferably, the thickness in the distal section 20 is reduced such that it becomes similar to the thickness exhibited by the proximal section 10.

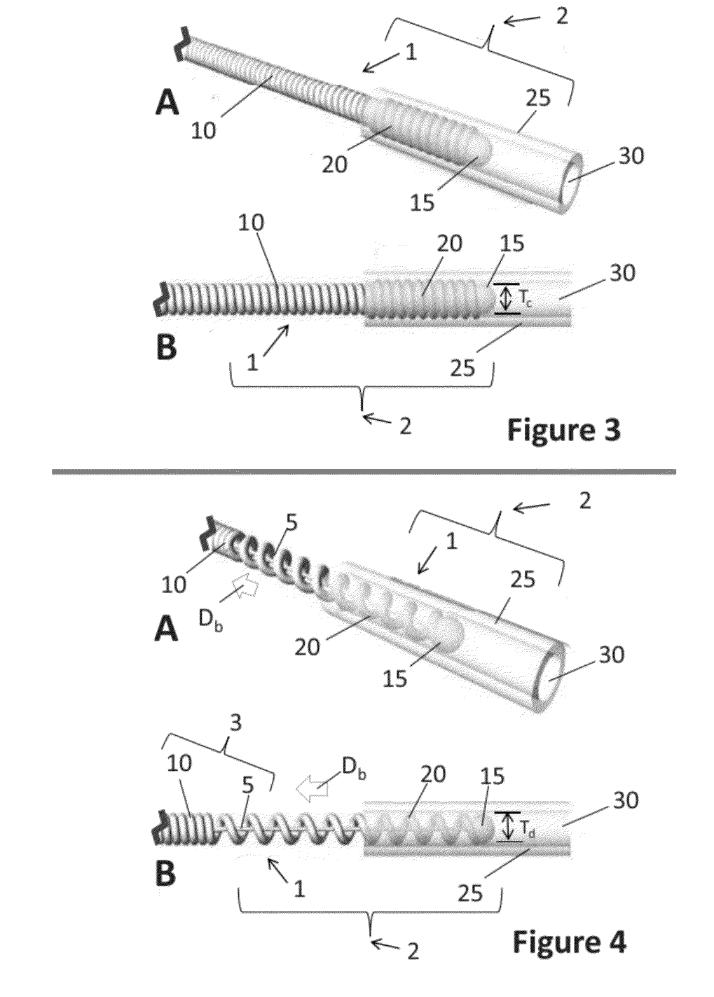

Referring now to FIGS. 3A & 3B, the quick release mechanism 1 and the medical device 25 make up a medical device assembly 2. The distal section 20 of the coiled wire 10 in its relaxed or engaged position interacts with an opening or lumen 30 in the medical device 25. The diameter of the lumen 30 and the thickness (T_{c}) of the distal section 20 is predetermined and sized such that the lumen 30 receives the outer diameter of the wire coiled at the distal section 20. In other words, the surface of the coiled wire 10 at its distal section 20 makes contact with the inner surface of the lumen 30 in the medical device 25 in a manner that causes the coiled wire 20 to securely hold the medical device 25.

Referring now to FIGS. 4A & 4B, the quick release mechanism 1 is caused to move to its detached position by pulling the coiled wire 10 backward, i.e., moving said coiled wire 10 in the proximal direction (D_{b}) relative to the vasculature of the patient. In this manner the overall thickness (T_{d}) of the distal section 20 becomes smaller, thereby, reducing contact between the outer surface of the coiled wire 10 at its distal section 20 and the inner surface of the lumen 30 in the medical device 25. This reduction in contact allows the medical device 30 to be released from the quick release mechanism 1 and to be deployed into the vasculature of the patient.

The medical device assembly 2 may comprise any metal, metal alloy, and/or polymeric materials known to one skilled-in-the-art. According to one aspect of the present disclosure, the quick release mechanism 1 and the medical device 25 that make up the medical device assembly 2 may comprise a super-elastic metal alloy, such as Nitinol, thereby, allowing for extended durability and flexibility. However, one skilled-in-the-art will understand that stainless steel or other metals and metal alloys may also be used with exceeding the scope of this disclosure. According to another aspect of this disclosure, at least a portion of the medical device assembly 2 may optionally have one or more surface treatments applied thereto, including but not limited to coatings, machining, and texturing.

The quick release mechanism 1 has two positions relative to the medical device 25. These positions are, namely, an engaged position and a detached position. In the engaged position, the quick release mechanism 1 engages the medical device and allows the pusher element 3 to move the quick release mechanism 1 and medical device 25 together as a medical device assembly 2, even after the medical device 25 is extruded from the end of the delivery catheter. In the detached position, the quick release mechanism 1 does not engage the medical device 25 and the medical device 25 is released or deployed at the selected target site. The medical device assembly 2 is designed such that the default position for the quick release mechanism 1 is the engaged position.

The switching between the engaged and detached positions is controlled by an operator, such as a physician or surgeon, through the manipulation of the inner core wire 5 and outer coil 10 of the pusher element 3. The movement of the inner core wire 5 in a distal direction along with movement of the outer coil 10 in a proximal direction relative to the vasculature of the patient causes the quick release mechanism 1 to switch from its engaged position to the detached position.

The quick release mechanism 1 as described herein can be adapted to be used with a variety of medical devices 25, including, but not limited to, embolic protection devices, occlusive devices, stents, and dilation balloons, among others. The medical device 25 may comprise any structure known to one skilled-in-the-art, including for example, occlusive devices of tubular structures, having braids, coils, a combination of braids and coils, or the like. The occlusive device may change shape during deployment, such as changing from a collapsed configuration to an expanded configuration. One example, among many examples, of a medical device 25 used with the quick release mechanism 1 of the present disclosure is a Nester® embolization coil (Cook Medical Incorporated, Bloomington, Ind.).

The delivery catheter used to deliver the medical device assembly 2 may be made of any material known to one skilled-in-the-art. Such material may include but not be limited a polyimide, polyether amide, nylon, polytetrafluoroethylene (PTFE), polyetheretherketone (PEEK), and mixtures or copolymers thereof. In its basic form, the catheter is a hollow elongated tube sized to receive the medical device assembly 2. The length of the delivery catheter may be any length necessary or desired to deploy the medical device 25 at the targeted site in the vasculature of a patient.

Another objective of the present disclosure is to provide a method of deploying a medical device 25 at a targeted site in the vasculature of a patient. This method generally comprises the steps of introducing the medical device assembly 2 described herein into the vasculature of the patient, wherein the quick release mechanism 1 is in its engaged position with the medical device 25; and then switching the quick release mechanism 1 to its detached position, thereby, deploying the medical device 25 at the targeted or desired site.

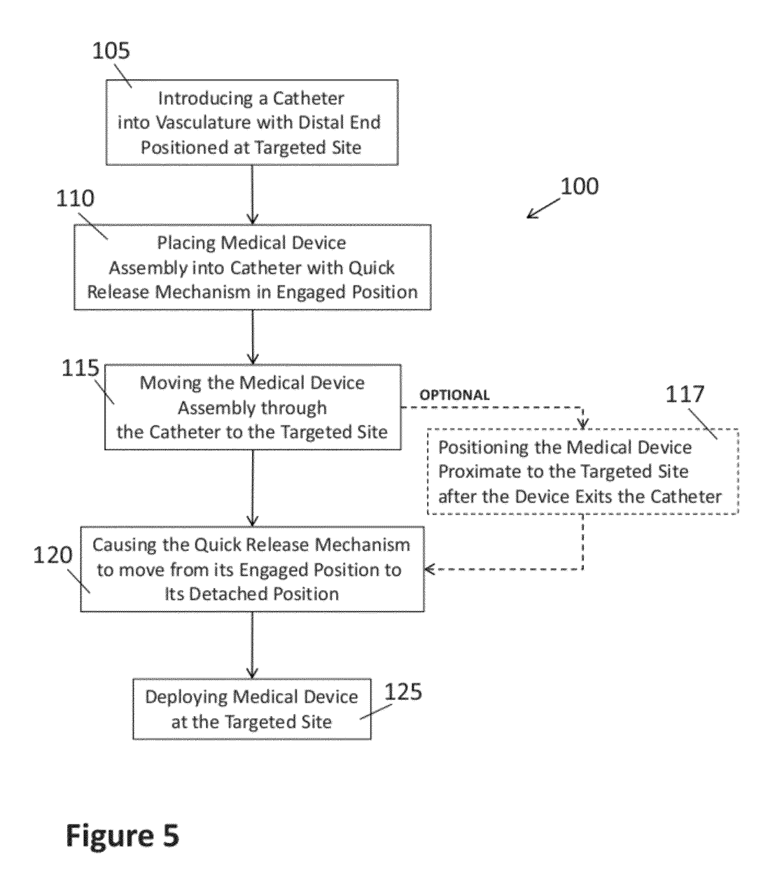

FIG. 5 depicts an example of a method 100 used to deploy a medical device 25 to a targeted or desired site in the vasculature of a patient. This method 100 generally comprises introducing 100 a catheter into the vasculature of a patient and positioning the distal end of said catheter at a desired or targeted site. A medical device assembly 2 including the aforementioned medical device 25 and quick release mechanism 1 is then placed 105 into the catheter. At this time, the quick release mechanism 1 is in its engaged position and securely holds the medical device 25. The pusher element 3 is then allowed to move 105 the medical device assembly 2 through the catheter to the targeted site. Optionally, the operator may further position 117 the medical device 25 proximate to the targeted site after the device 25 exits the catheter. The operator then causes the quick release mechanism 1 to move 120 from its engaged position to its detached position. The quick release mechanism 1 transitions from the engaged position to the detached position upon the operator continuing to move the inner core wire 5 in a proximal direction relative to the vasculature of the patient, while moving the outer coil 10 of the pusher element 3 in the opposite direction (e.g., in a distal direction relative to the vasculature of the patient). This combined motion causes the coiled wire at the distal section 20 to stretch such that contact between the coil 20 and the medical device 25 is reduced, thereby, allowing the medical device 25 to be deployed 125 into the vasculature of the patient at the targeted site.

The present disclosure provides a quick release mechanism 1 that securely holds medical device 25 during the deployment of the medical device 25. The quick release mechanism 1 also allows for the easy and reliable detachment of the medical device 25 once the device 25 is properly positioned proximate to the targeted site. The quick release mechanism 1 of the present disclosure provides the operator (e.g., physician) with improved control over the medical device 25 during its deployment, and allows the operator to position and even reposition the medical device 25 at the targeted site before detachment. One skilled-in-the-art will understand that the quick release mechanism 1 of the present disclosure is readily adaptable for use with a wide variety of medical devices.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A medical device assembly having a quick release mechanism for use by an operator in deploying a medical device at a targeted site in a body vessel, the medical device assembly comprising:
a quick release mechanism including a pusher element having a distal end, the pusher element including an inner core wire and an outer coil; the inner core wire and outer coil coupled together at the distal end; the outer coil having a proximal section and a distal section; the outer coil having an enlarged overall thickness in the distal section; and
a medical device having a proximal part and a distal part; the proximal part having an opening sized to receive and to detachably engage the distal section of the outer coil;
wherein the movement of the outer coil opposite to the movement of the inner core wire reduces the thickness of the outer coil in the distal section, thereby, allowing the medical device to detach from the wire guide for deployment in the body vessel.

2. The medical device assembly of claim 1, wherein the quick release mechanism has an engaged position and a detached position; the distal section of the outer coil having a thickness (T_{c}) when in the engaged position and a thickness (T_{d}) when in the detached position; wherein the thickness T_{c} is greater than the thickness T_{d}.

3. The medical device assembly of claim 2, wherein in the engaged position the distal section of the outer coil engages the medical device.

4. The medical device assembly of claim 2, wherein in the detached position the distal section of the outer coil does not substantially engage the medical device.

5. The medical device assembly of claim 1, wherein the operator reduces the thickness of the outer coil in the distal section by moving the inner core wire in a distal direction relative to the body vessel, while also moving the outer coil in a proximal direction relative to the body vessel.

6. The medical device assembly of claim 1, wherein at least one of the quick release mechanism and medical device are made from a metal, a metal alloy, a polymeric material, or a combination thereof.

7. The medical device assembly of claim 6, wherein the metal alloy is Nitinol.

8. The medical device of claim 1, wherein the medical device is one selected from the group of embolic protection devices, occlusive devices, stents, and dilation balloons.

9. The medical device of claim 8, wherein the occlusive device is an embolization coil.

10. A method for use by an operator in deploying a medical device at a targeted site in a body vessel, the method comprising the steps of:
introducing a catheter having a distal end and a proximal end into a body vessel;
the distal end being positioned proximate to a targeted site within the vessel;
placing a medical device assembly into the proximal end of the catheter; the medical device assembly comprising a medical device and a quick release mechanism having an engaged position in which the medical device and quick release mechanism are engaged and a detached position in which the medical device and quick release mechanism are not engaged;
moving the medical device assembly through the catheter to the targeted site in the vessel with the quick release mechanism in the engaged position;
causing the quick release mechanism to move from the engaged position to the detached position; and
deploying the medical device at the targeted site in the vessel.

11. The method of claim 10, wherein the step of placing a medical device assembly into a catheter uses a medical device assembly having a quick release mechanism that includes a pusher element with a distal end, the pusher element having an inner core wire and an outer coil; the inner core wire and outer coil being coupled together at the distal end; the outer coil having a proximal section and a distal section; the outer coil having an enlarged overall thickness in the distal section; and a medical device having a proximal part and a distal part; the proximal part having an opening sized to receive and to detachably engage the distal section of the outer coil.

12. The method of claim 11, wherein the step of causing the quick release mechanism to move from the engaged position to the detached position, further includes the operator reducing the thickness of the outer coil in the distal section by moving the inner core wire in a distal direction relative to the body vessel, while also moving the outer coil in a proximal direction relative to the body vessel.

13. The method of claim 11, wherein the method further comprises the operator positioning the medical device proximate to the targeted site after the device exits the distal end of the catheter and prior to causing the quick release mechanism to move from the engaged position to the detached position.

14. The method of claim 10, wherein the step of placing a medical device assembly into a catheter uses a medical device assembly having a medical device selected as one from the group of embolic protection devices, occlusive devices, stents, and dilation balloons.
